(19) United States
(12) Patent Application Publication
(10) Pub. No.: US 2015/0086616 A1
(43) Pub. Date: Mar. 26, 2015

(54) METHOD FOR THE PREVENTION AND TREATMENT OF ALZHEIMER'S DISEASE

(71) Applicant: Steven Lehrer, New York, NY (US)

(72) Inventor: Steven Lehrer, New York, NY (US)

(21) Appl. No.: 14/490,042

(22) Filed: Sep. 18, 2014

Related U.S. Application Data
(60) Provisional application No. 61/880,728, filed on Sep. 20, 2013.

(51) Int. Cl. A61K 9/00; A61K 45/06; A61K 38/28; A61K 31/196

(52) U.S. Cl. CPC A61K 9/0043; A61K 31/196; A61K 45/06; A61K 38/28
USPC 424/450; 514/567; 424/173.1; 514/6.5; 424/134.1

(57) ABSTRACT

Oral non-steroidal anti inflammatory drugs (NSAIDs) are not an effective treatment of Alzheimer's disease, because the brain dose is too low. Nasal delivery of NSAIDs such as ibuprofen, flurbiprofen, indomethacin, diclofenac, or naproxen, which inhibit the enzymes cyclooxygenase-1 (cox-1) and cox-2, are used to prevent and/or treat Alzheimer's disease, a low grade brain inflammation. The large amount of nasal NSAIDs (e.g., ibuprofen, flurbiprofen, indomethacin, diclofenac, or naproxen) that reaches the brain far exceeds that from an oral dose. Low-molecular-weight lipophilic drugs, such as ibuprofen, naproxen, indomethacin, diclofenac, and flurbiprofen, are readily absorbed into the brain by the intranasal route. Alzheimer's starts in the entorhinal cortex, which is closely connected to the olfactory nerves, and spreads anatomically in a defined pattern. Therefore, a nasal NSAID would readily reach the region of the brain where it acts therapeutically.

This application claims priority to U.S. Provisional Application 61/880,728, filed Sep. 20, 2013. The entire disclosure of the application is incorporated by reference herein.

### FIELD OF THE INVENTION

The present invention relates to the field of Alzheimer's disease. Specifically, compositions and methods for treating, inhibiting, and/or preventing Alzheimer's disease are disclosed.

### BACKGROUND OF THE INVENTION

Several publications and patent documents are cited throughout the specification in order to describe the state of the art to which this invention pertains. Each of these citations is incorporated herein by reference as though set forth in full.

Although several initially promising agents have been developed to reverse or at least slow the decline of cognitive function in Alzheimer's patients, successive clinical trials have failed. The most advanced agents are monoclonal antibodies directed to certain forms of Amyloid β (Aβ) and Tau protein. Trials are now being conducted in mild Alzheimer's disease, in prodromal Alzheimer's, and in subjects with Aβ plaques and minor memory complaints, but no deficits on standard cognitive tests. Other amyloid-based approaches include anti-aggregation compounds, inhibitors of BACE (β-secretase enzyme), and either modulators or inhibitors of γ-secretase. Tau-protein-lowering approaches include anti-aggregation and immunotherapy (Selkoe, D. J. (2013) Cell 154(2):468). Yet the amyloid β plaques and tau protein tangles, hallmarks of the pathology, may be a result of the disease process, rather than a cause. Indeed, Alzheimer's disease pathology is common in older persons without cognitive impairment (Bennett et al. (2012) Ann. Neurol., 72(4):599-609). Accordingly, new therapeutic methods for the inhibition and/or treatment of Alzheimer's disease are needed.

### SUMMARY OF THE INVENTION

In accordance with the instant invention, methods of treating, inhibiting (e.g., slowing or reducing), preventing, and/or inhibiting the onset of a central nervous disease or disorder in a subject are provided. In a particular embodiment, the central nervous disease or disorder is Alzheimer's disease. In a particular embodiment, the method comprises administering a composition comprising at least one non-steroidal anti-inflammatory drug (NSAID) and, optionally, at least one pharmaceutically acceptable carrier to the nasal cavity of the subject. In a particular embodiment, the method comprises administering at least diclofenac to a subject. The method may further comprise the administration of at least one other Alzheimer's disease therapy.

### DETAILED DESCRIPTION OF THE INVENTION

Some Alzheimer's researchers are skeptical of the amyloid hypothesis and believe that the malady results from low grade inflammation of the brain (Wyss-Coray et al. (2012) Cold Spring Harb. Perspect. Med., 2(1):a006346). The 1.8/1 female to male ratio indicates that the inflammation is autoimmune in nature (Lehrer et al. (2014) Am. J. Alzheimer's Dis. Other Demen., doi:1533317514545478). Implication of inflammation in Alzheimer's is not surprising, since inflammation underlies many diseases of aging including, for example: neurodegenerative (McGeer et al. (2004) Ann. NY Acad. Sci., 1035:104-116), osteoarthritis and rheumatoid arthritis (McGeer et al. (2004) Ann. NY Acad. Sci., 1035:104-116), cardiovascular (Kalogeropoulos et al. (2010) J. Am. Coll. Cardiol., 55(19):2129-2137), and cancer (Coussens et al. (2002) Nature 420(6917):860-867). In addition, the apoE4 allele, which increases the risk of Alzheimer's disease, is associated with significantly greater systemic and brain elevations of the pro-inflammatory cytokines TNFα and IL-6, as compared with their apoE3 counterparts, indicating an isoform-specific effect of the immunomodulatory properties of apoE (Lynch et al. (2003) J. Biol. Chem., 278(49):48529-48533).

Epidemiological observation indicates that long-term oral administration to patients suffering from rheumatoid arthritis of the non-steroidal anti-inflammatory drug (NSAID) ibuprofen may result in some reduced risk and delayed onset of Alzheimer's disease, although oral ibuprofen, flurbiprofen, and other NSAIDs are not an effective treatment (Imbimbo et al. (2010) Front Aging Neurosci., 2:19; Osborne, R. (2008) Nat. Biotech., 26(8):841-843). Indeed, oral ibuprofen is an ineffective treatment because the oral dose to the brain is too low. In stark contrast and in accordance with the instant invention, nasal delivery of NSAIDs such as ibuprofen, naproxen, indomethacin, diclofenac, flurbiprofen and other cyclooxygenase-1 (cox-1) and cox-2 inhibitors inhibit, treat, and/or prevent Alzheimer's disease.

Ibuprofen, naproxen, diclofenac, and flurbiprofen, which are highly lipophilic, readily cross the blood brain barrier (Parepally et al. (2006) Pharm. Res., 23(5):873-881). However, the amount of ibuprofen, naproxen, diclofenac, or flurbiprofen that reaches the brain after an oral dose is small and the distribution is extremely poor. For example, in one study, after multiple oral doses of ibuprofen the concentration in fat was 58.4 μg/g tissue, while brain concentration was 0.4 μg/g (Adams et al. (1969) Toxicol. Appl. Pharmacol., 15(2):310-330).

The NSAID brain dose will be significantly increased by delivering the drug intranasally (Lehrer, S. (2014) Am. J. Alzheimers Dis. Other Demen., 29(5):401-403). Notably, nasal formulations of NSAIDs have been generated, albeit for other purposes (U.S. Patent Application Publication No. 2001/0055569). Nasal drug delivery that exploits the olfactory and trigeminal neuronal pathways to convey drugs to the brain is being widely explored by pharmaceutical companies. Low-molecular-weight and lipophilic drugs, such as ibuprofen, naproxen, diclofenac, or flurbiprofen, are readily absorbed into the brain by the intranasal route (Bahadur et al. (2012) Expert Opin. Drug Deliv., 9(1):19-31). Indeed, intranasal insulin and other compounds have been tested as a treatment for Alzheimer's disease (Freiherr et al. (2013) CNS Drugs 27(7):505-514; WO 2011/054759). 1H-indole-3-glyoxylamide, an sPLA2 inhibitor, can also be administered to the lung or as a nasal spray for the prevention and treatment of Alzheimer's disease (U.S. Pat. No. 5,654,326). Other methods for treating Alzheimer's disease include the administration of compounds which inhibits Aβ peptide polymerization along with a complementing NSAID (WO 2014/066318). However, the use of NSAID monotherapy has not been taught, particularly in view of its previous failure by non-nasal routes (Jaturapatporn et al. (2012) Cochrane Database Syst. Rev., 2:CD006378).

Alzheimer's disease starts in the entorhinal cortex, which is closely connected to the olfactory nerves, and spreads anatomically in a defined pattern (Liu et al. (2012) PLoS One 7(2):e31302). Therefore, nasal NSAIDs would readily reach the region of the brain where they are therapeutic.

The NSAIDs of the instant invention, e.g., ibuprofen, naproxen, diclofenac, or flurbiprofen, may be contained in liposomes, microspheres, nano-emulsions, or nanoparticles. For example, microsphere encapsulation of thyrotropin releasing hormone has proven effective for nasal administration (Kubek et al. (2009) Neurotherapeutics 6(2):359-371; U.S. Pat. No. 7,537,787; U.S. Pat. No. 3,941,127; U.S. Pat. No. 4,849,228; U.S. Pat. No. 5,171,812; U.S. Pat. No. 5,360,610; U.S. Pat. No. 5,487,739; U.S. Pat. No. 5,652,220; U.S. Pat. No. 6,303,134; U.S. Pat. No. 6,491,939; U.S. Pat. No. 6,699,491; U.S. Pat. No. 7,229,635).

Other non-steroidal anti-inflammatory drugs (NSAIDs) and their enantiomers also lower the risk of Alzheimer's disease and can be used nasally as a treatment. Examples of NSAIDs include, without limitation:
Salicylates: Aspirin (acetylsalicylic acid), Diflunisal, Salsalate
Propionic acid derivatives: Ibuprofin, Naproxen, Flubiprofen, Dexibuprofen, Fenoprofen, Ketoprofen, Dexketoprofen, Oxaprozin, Loxoprofen, Tarenflurbil
Acetic acid derivatives: Indomethacin, Tolmetin, Sulindac, Etodolac, Diclofenac, Nabumetone. Nasal ketorolac is used for management of moderate to severe pain.
Enolic acid (Oxicam) derivatives: Piroxicam, Meloxicam, Tenoxicam, Droxicam, Lornoxicam, Isoxicam
Fenamic acid derivatives (Fenamates): Mefenamic acid, Meclofenamic acid, Flufenamic acid, Tolfenamic acid
Selective COX-2 inhibitors: Celecoxib, Rofecoxib, Valdecoxib, Parecoxib, Lumiracoxib, Etoricoxib, Firocoxib, Paracetamol (acetaminophen)
Sulphonanilides: Nimesulide
Others: Licofelone, H-harpagide in Figwort or Devil's Claw, Lysine clonixinate, TREM-2 mediators
   In a particular embodiment, the NSAID is an inhibitor of COX-1 and/or COX-2. In a particular embodiment, the NSAID inhibits COX- and COX-2 to the same degree. In a particular embodiment, the NSAID is selected from the group consisting of ibuprofen, indomethacin, naproxen, flubiprofen, and diclofenac or the group consisting of ibuprofen, naproxen, flubiprofen, and diclofenac. In a particular embodiment, the NSAID is diclofenac.

Animal model data as well as recently published trial data indicate that cox-1 plays an important role in early Alzheimer's pathogenesis and is a potential target for early intervention (Frautschy, S. A. (2010) Neurobiol. Dis., 38(3):492-494; Dargahi et al. (2011) J. Mol. Neurosci., 45(1):10-21; Choi et al. (2013) J. Neurochem., 124(1):59-68). Flurbiprofen, which inhibits both cox 1 and cox 2, has 12½ times the potency of ibuprofen (Nozu, K. (1978) Biochim. Biophys Acta, 529(3):493-496). Accordingly, a smaller nasal dose of flurbiprofen would be required. In a particular embodiment of the instant invention, a nasal dose of at least 600 mg or about 600 to about 800 mg ibuprofen (or equivalent thereof) is administered. This dose is at the upper level of acceptability. In a particular embodiment, flurbiprofen is administered at a dose of at least about 10 mg, at least about 100 mg, or about 10 to about 200 mg.

Notably, R-flurbiprofen (tarenflurbil), an enantiomer of flurbiprofen, failed a phase III clinical trial for the treatment of Alzheimer's disease. However, R-Flurbiprofen is devoid of any direct cyclooxygenase inhibition. In stark contrast, the S-enantiomer of flurbiprofen is a cyclooxygenase inhibitor (Green et al. (2009) JAMA 302(23):2557-2564). Therefore, the S-enantiomer may be used as an effective Alzheimer's therapy.

In a particular embodiment, diclofenac is used in place of or in addition to flurbiprofen. Diclofenac is one of the most potent NSAIDs. Furthermore, ophthalmic diclofenac has more anti-inflammatory action and produces less burning sensation than flurbiprofen (Diestelhorst et al. (1996) J. Cataract Refract Surg., 22(Suppl 1):788-793). In a particular embodiment, diclofenac is administered at a dose of at least about 1 mg, at least about 10 mg, or about 1 to about 200 mg or about 10 to 200 mg.

The efficacy of treatment can be assessed by the appearance of improvement or no deterioration, or slower than expected deterioration in at least one of the assessments selected from the group consisting of the Alzheimer's Disease Assessment Scale-cognitive subscale (ADAS-cog), the Alzheimer's Disease Cooperative Study-Activities of Daily Living (ADCS-ADL) Inventory and Clinician's Interview-Based Impression of Change Plus Version (CIBIC-plus).

While the instant invention describes the use of nasal NSAIDs for the treatment of Alzheimer's disease, the methods of the instant may be used to treat, inhibit, and/or prevent other central nervous system diseases, particularly those associated with aging, including, without limitation: frontotemporal dementia, Parkinson's disease, amyotrophic lateral sclerosis, the Parkinson-dementia complex of Guam, all of the tauopathies, and age-related macular degeneration.

Nasal NSAIDs, especially ibuprofen, flurbiprofen, diclofenac, and naproxen, may be used to prevent Alzheimer's disease in high risk subjects, that is, persons carrying one or two copies of the apoE4 allele. Other high risk subjects include, without limitation, smokers, obese people, and diabetics.

The nasal NSAIDs may also be combined with another anti-inflammatory or other Alzheimer's drugs, such as a TNF inhibitor (e.g., etanercept), bexarotene, thiazolidinedione, or anti-diabetics such as rosiglitazone or pioglitazone. In a particular embodiment, the anti-diabetic agent is a biguanide or a glitazone. Biguanides include, without limitation, metformin, phenformin, buformin, proguanil, and pharmaceutically acceptable salts or esters thereof. Glitazones (also known as thiazolidinediones (TZDs)) include, without limitation, rosiglitazone (Avandia®), pioglitazone (Actos®), troglitazone (Rezulin®), and pharmaceutically acceptable salts or esters thereof. In a particular embodiment, the anti-diabetic agent is a sulphonylurea drug (e.g., acetohexamide, chlorpropamide, tolbutamide, glipizide, glyburide), a sulfonamide (e.g., tolazemide), an alpha-adrenergic antagonist (e.g., phentolamine), or pharmaceutically acceptable salts or esters thereof.

Nasal insulin is also beneficial for Alzheimer's patients. In a particular embodiment of the instant invention, a nasal NSAID is administered with a preservative-free nasal insulin (e.g., U.S. Patent Application Publication No. 2014/0179597). The agents may be delivered via a single composition (e.g., spray).

As stated hereinabove, the NSAIDs of the instant invention are administered nasally. In a particular embodiment, the NSAIDs are administered to the upper third of the nasal cavity of the subject, e.g., the olfactory area and epithelium in the upper third of the nasal cavity as opposed to pulmonary capillaries. Without being bound by theory, the olfactory and/or trigeminal neuronal pathway may be exploited to convey the drugs to the brain. In a particular embodiment, the NSAIDs of the instant invention are administered nasally via pressurized aerosol, aqueous pump spray, powder, gel, drops, or other method (Thorsson et al. (1999) Br. J. Clin. Pharmacol., 47:619-624). Nasal solutions are generally prepared to be similar to nasal secretions and are generally isotonic and slightly buffered to maintain a pH of about 5.5 to about 6.5, although pH values outside of this range can additionally be used (e.g., a pH of about 5 to about 8). Antimicrobial agents or preservatives can also be included in the formulation.

The compositions of the instant invention may be delivered to the nasal cavity as a powder or liquid nasal spray, aerosol spray, nose drop, suspension, or a gel or film. Nasal drug delivery of the compositions may be performed using, without limitation, pump sprays, pump-atomizers, droppers, squeeze bottles, pressurized sprays, nebulizers, aerosolizes, and inhalers. The delivery device may be metered and/or deliver a unit dose. In a particular embodiment, the composition is delivered as an aerosol spray, e.g., using a pressurized container and a suitable propellant (e.g., a gas such as nitrogen, air, or carbon dioxide, or a fluid such as 1,1,1,2-tetrafluoroethane, 1,1,1,2,3,3,3-heptafluoropropane, dichlorodifluoromethane, trichlorofluoromethane, dichlorotetrafluoroethane, fluorocarbons, fluorinated chlorinated hydrocarbons, hydrochlorofluorocarbons, hydrochlorocarbons, hydrocarbon ethers, and hydrocarbons (e.g., n-butane, propane, isopentane, or a mixture thereof)).

The compositions of the instant invention may further comprise agents which facilitate the nasal delivery of NSAIDs. The agents may be delivered simultaneously and/or sequentially (e.g., before and/or after) the administration of the NSAID. Examples of such agents include, without limitation, hyaluronidase, protease inhibitors, vasoconstrictor, and surfactants.

As explained above, the pharmaceutical formulations for use in the therapies of the present invention may include, as optional ingredients, pharmaceutically acceptable carriers, diluents, solubilizing or emulsifying agents, and salts of the type that are well-known in the art. Examples of such substances include normal saline solutions, such as physiologically buffered saline solutions, and water. The compounds of the present invention can be administered as a dry powder or in an aqueous solution. In a particular embodiment, a physiologically-acceptable buffered saline solution may be used.

Dry aerosol in the form of finely divided solid particles of compounds that are not dissolved or suspended in a liquid are also useful in the practice of the present invention. The compounds may be in the form of dusting powders and comprise finely divided particles having an average particle size of between about 1 and 5 microns, or between 2 and 3 microns. Finely divided particles may be prepared by pulverization and screen filtration using techniques well known in the art. The particles may be administered by inhaling a predetermined quantity of the finely divided material into the nasal cavity, which can be in the form of a powder.

An aerosol formulation used for nasal administration may be an aqueous solution designed to be administered to the nasal passages in drops or sprays. Nasal solutions are generally prepared to be similar to nasal secretions and are generally isotonic and slightly buffered to maintain a pH of about 5.5 to about 6.5, although pH values outside of this range can additionally be used (e.g., a pH of about 5 to about 8). Antimicrobial agents or preservatives can also be included in the formulation. The compositions of the instant invention may further comprise a stabilizer. The stabilizer may be water. The compositions may further comprise a cosolvent (e.g., a cosolvent comprising ethanol). Aerosol formulations can also include other components, for example, ethanol, isopropanol, propylene glycol, as well as surfactants or other components such as oils and detergents (see, e.g., Remington's Pharmaceutical Sciences, 1990; U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol may comprise a solution of an active ingredient such as an NSAID in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient anti-diabetic agent and a propellant and can include any combination of solvents and preservatives or antioxidants.

An aerosol formulation can also be a dispersion or suspension. A suspension aerosol formulation may contain a suspension of an NSAID and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient NSAID. The surfactant can be nonionic, anionic or cationic. One example of an emulsion can include, for example, ethanol, surfactant, water and propellant. Another example of an emulsion can include, for example, vegetable oil, glyceryl monostearate and propane.

In order to inhibit, treat and/or prevent Alzheimer's disease, an NSAID may be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of an NSAID required to obtain an effective dose in a subject depends on many factors, including the severity of the Alzheimer's disease being treated, the age, weight and general health of the subject, as well as the number of treatments to be administered. In view of these factors, the skilled artisan would adjust the particular dose so as to obtain an effective dose for treating an individual having Alzheimer's disease.

When administering high doses of an NSAID, one skilled in the art can monitor for any possible adverse side effects. Methods of monitoring adverse side effects of an NSAID are known in the art. One of skilled in the art can monitor for any adverse side effects and, if necessary, adjust the dosage to minimize adverse side effects while optimizing the effectiveness of treating or preventing Alzheimer's disease.

The concentration of an anti-diabetic agent in a particular formulation will depend on the mode and frequency of administration. A given daily dosage can be administered in a single dose or in multiple doses so long as the NSAID concentration in the formulation results in the desired daily dosage. One skilled in the art can adjust the amount of an NSAID in the formulation to allow administration of a single dose or in multiple doses that provide the desired concentration of an NSAID over a given period of time.

When an NSAID is administered in combination with one or more other anti-Alzheimer's disease agent, the NSAID and other agent can be co-administered in the same formulation. Alternatively, the NSAID and other agent can be administered simultaneously in separate formulations. In addition, the NSAID and other agent can be administered in separate formulations, where the separate formulations are not administered simultaneously but are administered during the same period of treatment, for example, during a daily or weekly period of treatment.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

### DEFINITIONS

The following definitions are provided to facilitate an understanding of the present invention:

The singular forms “a,” “an,” and “the” include plural referents unless the context clearly dictates otherwise.

As used herein, the terms “host,” “subject,” and “patient” refer to any animal, particularly mammals including humans.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., Alzheimer's disease) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to inhibit Alzheimer's disease progression in a subject.

What is claimed:

1. A method for inhibiting, treating, or preventing a disease of the central nervous system comprising administration to a subject in need thereof of an effective amount of non-steroidal anti-inflammatory drug (NSAID) to achieve a therapeutically effective brain level, wherein said NSAID is administered nasally.

2. The method of claim 1, wherein said disease is Alzheimer's disease.

3. The method of claim 1, wherein said NSAID is selected from the group consisting of ibuprofen, indomethacin, naproxen, flubiprofen, and diclofenac.

4. The method of claim 3, wherein the NSAID is diclofenac.

5. The method of claim 1, wherein achieving a therapeutically effective brain level of said NSAID comprises administering a dose of a pharmaceutical formulation of the NSAID into a single nostril or both nostrils.

6. The method of claim 4, wherein a dose comprises at least 10 mg to 200 mg of diclofenac.

7. The method of claim 1, wherein the NSAID is in the form of a dry powder or dissolved in solution.

8. The method of claim 1, wherein the NSAID is encapsulated in liposomes, microspheres, nano-emulsions, or nanoparticles.

9. The method of claim 1, wherein the pharmaceutical formulation of said NSAID is administered at least once per day.

10. The method of claim 1, wherein a nozzle of a spray device is used to administer a unit dose of the NSAID pharmaceutical preparation by ejection through a nostril of a mammal to the olfactory region.

11. The method of claim 1, wherein said disease of the central nervous system is selected from the group consisting of Parkinson's disease, amyotrophic lateral sclerosis, the Parkinson-dementia complex of Guam, tauopathy, and age-related macular degeneration.

12. The method of claim 2, wherein the treatment is determined by improvement or no deterioration, or slower than expected deterioration in at least one of the assessments selected from the group consisting of the Alzheimer's Disease Assessment Scale-cognitive subscale (ADAS-cog), the Alzheimer's Disease Cooperative Study-Activities of Daily Living (ADCS-ADL) Inventory and Clinician's Interview-Based Impression of Change Plus Version (CIBIC-plus).

13. The method of claim 2, further comprising the administration of anti-amyloid antibody, etanercept, bexarotene, or an anti-diabetic agent.

14. The method of claim 2, wherein the subject is a high risk subject.

15. The method of claim 14, wherein said high risk subject is a smoker, diabetic, obese, or carries one or two copies of the apoE4 allele.

16. The method of claim 2, further comprising the nasal administration of insulin.

17. The method of claim 2, consisting of nasal administration to a subject in need thereof of an effective amount of non-steroidal anti-inflammatory drug (NSAID) to achieve a therapeutically effective brain level.

18. The method of claim 17, wherein the NSAID is diclofenac.
